SEQUENCE LISTING (1 sequence)

SEQ ID NO: 1 - protein, 9 aa, Unknown
  Description of Unknown “LAGLIDADG” family peptide motif sequence
LAGLIDADG

(19) United States
(12) Patent Application Publication
(10) Pub. No.: US 2012/0252122 A1
(43) Pub. Date: Oct. 4, 2012

(54) METHODS AND COMPOSITIONS FOR INCREASING PRODUCTION OF INDUCED PLURIPOTENT STEM CELLS (IPSCS)

(75) Inventor: Josee LAGANIERE, El Cerrito, CA (US)

(73) Assignee: Sangamo BioSciences, Inc.

(21) Appl. No.: 13/326,882

(22) Filed: Dec. 15, 2011

Related U.S. Application Data
(60) Provisional application No. 61/460,687, filed on Jan. 5, 2011.

(51) Int. Cl. C12N 15/85; C12N 5/0789; C12N 9/16; C12N 5/071

(52) U.S. Cl. 435/455; 435/377; 435/196

(57) ABSTRACT

Disclosed herein are methods and compositions for increasing yield of induced pluripotent stem cells (iPSCs).

### CROSS-REFERENCE TO RELATED APPLICATIONS

The present application claims the benefit of U.S. Provisional Application No. 61/460,687, filed Jan. 5, 2011, the disclosure of which is hereby incorporated by reference in its entirety.

### STATEMENT OF RIGHTS TO INVENTIONS MADE UNDER FEDERALLY SPONSORED RESEARCH

Not applicable.

### TECHNICAL FIELD

The present disclosure is in the field of cell culture and genome engineering, particularly increasing production of induced pluripotent stem cells (iPSCs).

### BACKGROUND

Stem cells are undifferentiated cells that exist in many tissues of embryos and adult mammals. Both adult and embryonic stem cells are able to differentiate into a variety of cell types and, accordingly, may be a source of replacement cells and tissues that are damaged in the course of disease, infection, or because of congenital abnormalities. (See, e.g., Lovell-Badge (2001) Nature 414:88-91; Donovan et al. (2001) Nature 414:92-97). Various types of putative stem cells exist which; when they differentiate into mature cells, carry out the unique functions of particular tissues, such as the heart, the liver, or the brain. Pluripotent stem cells are thought to have the potential to differentiate into almost any cell type, while multipotent stem cells are believed to have the potential to differentiate into many cell types (Robertson (1997) Meth. Cell Biol. 75:173; and Pedersen (1994) Reprod. Fertil. Dev. 6:543).

Induced pluripotent stem cells (iPSCs) have been generated from differentiated somatic cells of diverse origin (e.g., endoderm-, mesoderm-, and ectoderm-tissue derived iPSCs) by the ectopic expression of reprogramming factors. See, Takahashi & Yamanaka (2006) Cell 126:663-676; Nakagawa et al, (2008) Nat. Biotechnol 26:101-106). These iPSCs, including human iPSCs, have been shown to share all the key characteristics of embryonic stem cells (ESCs) and can be generated from cells isolated from human patients with specific diseases (see for example Dimos et al, (2008) Science 321:1218-1221). However, the efficiencies of producing of iPSCs (including genomically modified iPSCs) using these standard methods alone can be low.

Additionally, procedures have now been developed that allow transdifferentiation of cells of a differentiated type into a second differentiated cell type where the differentiated cells are treated with lineage specific factors of the second cell type which causes direct differentiation from the first type to the second type (see Vierbuchen et al, (2010) Nature 463(7284): 1035-1041). However, efficiency of these methods may not be optimal.

Thus, there remains a need for compositions and methods for increasing the efficiency of iPSC production from differentiated somatic cells.

### SUMMARY

Described herein are methods and compositions for producing iPSCs, in particular for increasing the yield and/or production of iPSCs from differentiated somatic cells. The methods involve the use of transient hypothermia, i.e. “cold shock,” to cells. In certain embodiments, the cells are genetically modified (e.g., using a nuclease) before, during or after cold shock treatment.

In one aspect, described herein is a method for increasing production of iPSCs from differentiated somatic cells (e.g., mammalian, particularly human cells) by subjecting the differentiated cells to cold shock; returning the cells to an appropriate (non-cold shock conditions) temperature following the cold shock, and producing iPSCs from the differentiated somatic cells. Methodology for iPSC production can be any method known in the art (e.g. Takashi and Yamanaka (2006) Cell 126: 663-676 or Yu et al, (2007) Science 318(5858): 1917-1920). In certain embodiments, the cold shock temperature is between 25° C. and 33° C. In certain embodiments, the cells are subjected to cold shock for between 1 hour and 4 days or any time therebetween. In some embodiments, the cold shock is performed during the transduction or introduction of iPSC factors. In other embodiments, the cold shock is performed immediately following the introduction of the iPSC factors.

In any of the methods described herein, the differentiated somatic cells may be fibroblasts, keratinocytes, peripheral blood cells or astrocytes. Furthermore, in any of the methods described herein, the cells are genetically modified, either before, during or after cold shock and/or production of iPSCs. In certain embodiments, the differentiated somatic cells are subject to cold shock in the presence of at least one substance that genetically modifies the differentiated somatic cell (e.g., a nuclease such as a zinc finger nuclease or Transcription activator like effector nuclease (TALEN)), and, following the cold shock, the modified cells are then reprogrammed into iPSCs using any suitable procedure.

Furthermore, any suitable nuclease may be employed for genomic modification. In certain embodiments, the nuclease comprises a zinc finger nuclease and/or a TALE protein (Transcription activator like), wherein the TALE comprises one or more engineered TALE binding domains. In any of the methods described herein, the nuclease(s) is(are) delivered to the cell via Integration Defective Lentiviral (IDLV) constructs (see for example United States Patent Publication 2009/0117617, incorporated herein by reference) or by integration competent lentiviral vectors. In another aspect, the nuclease(s) is(are) delivered to the cell via an adenoviral vector or an adenoviral associated vector (AAV).

Thus, provided herein is a method of increasing the production of iPSCs from differentiated somatic cells, the method comprising the steps of: introducing one or more expression constructs that express a nuclease(s) into any of a population of differentiated somatic cells (e.g., fibroblasts), incubating the cells under cold shock conditions such that the nuclease is expressed; culturing the cells in an appropriate temperature; producing iPSCs from the modified cells such that the proportion of differentiated somatic cells that are converted (reprogrammed) into iPSCs is higher than in cells not subject to cold-shock treatment. In any of the methods described herein, the nuclease may comprise, for example, a non-naturally occurring DNA-binding domain (e.g., an engineered zinc finger protein, a TAL-effector nuclease fusion protein, or an engineered DNA-binding domain from a homing endonuclease). In certain embodiments, the nuclease is a zinc finger nuclease (ZFN) or pair of ZFNs. In other embodiments, the nuclease is a TAL-effector domain nuclease fusion protein.

Any of the methods may further comprise introducing an exogenous sequence into the host cell such that the nuclease mediates targeted integration of the exogenous sequence into the genome. In certain embodiments, the exogenous sequence is introduced at the same time as the nuclease(s). In some aspects, the exogenous sequence may comprise a reporter gene. In certain embodiments, the methods further comprising isolating the cells expressing the reporter gene. In any of the methods described herein, the genomic modification is a gene disruption and/or a gene addition.

In another aspect of the invention, the cold shock is performed during a transdifferentiation process (see T. Vierbuchen, ibid). In this way, increased production of cells transdifferentiated (e.g. from a fibroblast into a neuron) are generated. The cold shock can be co-administered with the delivery of the transdifferentiation factors (lineage-specific transcription factors) or immediately following delivery of these factors.

In another aspect, the invention provides kits for increasing production of iPSCs or increasing transdifferentiation efficiency. The kits typically include one or more nucleases that bind to a target site, optional cells containing the target site(s) of the nuclease and instructions for introducing the nucleases into the cells and cold shocking the cells to increase yield of iPSCs or transdifferentiated cells. In certain embodiments, the kits comprise at least one construct with the target gene and a known nuclease capable of cleaving within the target gene. Such kits are useful for optimization of cleavage conditions in a variety of varying host cell types. Other kits contemplated by the invention may include a known nuclease capable of cleaving within a known target locus within a genome, and may additionally comprise a donor nucleic acid encoding a reporter gene.

### DETAILED DESCRIPTION

Described herein are compositions and methods to increase production of iPSCs from differentiated somatic cells and for increasing the efficiency of transdifferentiation. In particular, the methods use transient hypothermia of the somatic cells for varying length of times. After the period of cold shock, the somatic cells are returned to a more appropriate temperature for conversion to iPSCs. Alternately, the cold shock may be applied during the conversion process. The somatic cells may be genetically modified, for example using one or more nucleases, before, during or after cold shock treatment. In certain embodiments, the cells are genetically modified during cold shock treatment. In other embodiments, the methods and compositions are used to increase the efficiency of transdifferentiation.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains (e.g., the recognition helix region) can be “engineered” to bind to a predetermined nucleotide sequence. The engineered region of the zinc finger is typically the recognition helix, particularly the portion of the alpha-helical region numbered −1 to +6. Backbone sequences for an engineered recognition helix are known in the art. See, e.g., Miller et al. (2007) Nat Biotechnol 25, 778-785. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528 and 2008/0131962, incorporated herein by reference in their entireties.

“Cold shock” refers to a shift in temperature wherein cells are placed in a hypothermic environment that is colder than optimal growth temperature. The cold shock temperature will depend on the cell type, in particular the temperature that is optimal for cell division to occur in that cell type. For mammalian cells, cold shock temperatures will typically be, 33° C., 32° C., 31° C., 30° C., 29° C., and 28° C. or even lower. Zebrafish cell lines are grown at 28° C., so an optimal cold shock temperature would be lower than 28° C., for example, lower than 25° C., 24° C., 23° C., 22° C., or even lower. Similarly, plant protoplasts divide at cooler temperatures than mammalian cells, and so a suitable cold shock temperature would necessarily be cooler than that used for mammalian cells.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP DNA-binding domain is fused to a cleavage domain, the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

Overview

Described herein are compositions and methods for increasing the yield (production) of iPSCs from differentiated cells, including genetically modified differentiated cells. In the methods described herein, the differentiated cells are subject to transient hypothermia, which increases the yield of iPSCs. The differentiated cells may be subject to genetic modification, for example, nuclease-mediated genomic modification before, during or after cold-shock.

Differentiated Cells

Any differentiated somatic cell may be used with the practice of the present disclosure to produce iPSCs. The cell types can be cell lines or natural (e.g., isolated) cells such as, for example, primary cells. Cell lines are available, for example from the American Type Culture Collection (ATCC), or can be generated by methods known in the art, as described for example in Freshney et al., Culture of Animal Cells, A Manual of Basic Technique, 3rd ed., 1994, and references cited therein.

Other non-limiting examples of cell types include cells that have or are subject to pathologies, such as cancerous cells and transformed cells, pathogenically infected cells, stem cells, fully differentiated cells, partially differentiated cells, immortalized cells and the like. Thus, cells can be isolated by methods known in the art. In certain embodiments, the differentiated cells are isolated from a subject with a particular pathology. In certain embodiments, the cells are fibroblasts. In other embodiments, the differentiated cells are keratinocytes, peripheral blood cells or astrocytes. See, Ruiz et al. (2010) PLoS One December 9; 5(12):e15526.

Prokaryotic (e.g., bacterial) or eukaryotic (e.g., yeast, plant, fungal, piscine and mammalian cells such as feline, canine, murine, bovine, porcine, non-human primate and human) cells can be used, with eukaryotic cells being preferred. Suitable mammalian cell lines include K562 cells, CHO (Chinese hamster ovary) cells, 293 cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells and HeLa cells, 293 cells (see, e.g., Graham et al. (1977) J. Gen. Virol. 36:59), and myeloma cells like SP2 or NS0 (see, e.g., Galfre and Milstein (1981) Meth. Enzymol. 73(B):3 46), rat C6 cells, and porcine Pk15 cells. Peripheral blood mononucleocytes (PBMCs) or T-cells can also serve as hosts.

Nucleases

Described herein are compositions, particularly nucleases, which are useful in integration of a peptide fusion inhibitor into a cell surface receptor (e.g., viral receptor) or disruption of the cell surface receptor to inhibit entry of macromolecules that bind to the cell surface receptor. In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

A. DNA-Binding Domains

In certain embodiments, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In certain embodiments, the nuclease comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain comprises a naturally occurring or engineered (non-naturally occurring) TAL effector DNA binding domain. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove, (2009) Science 326:1501 and Boch et al (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and IG binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN) exhibiting activity in a yeast reporter assay (plasmid based target). Christian et al ((2010)<Genetics epub 10.1534/genetics.110.120717). See, also, U.S. Patent Publication No. 20110301073, incorporated by reference in its entirety.

In certain embodiments, the DNA binding domain comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635;7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, DNA domains (e.g., multi-fingered zinc finger proteins) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The zinc finger proteins described herein may include any combination of suitable linkers between the individual zinc fingers of the protein. In addition, enhancement of binding specificity for zinc finger binding domains has been described, for example, in co-owned WO 02/077227.

Selection of target sites; ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

B. Cleavage Domains

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Nat'l Acad Sci USA 93(3):1156-1160. More recently, ZFNs have been used for genome modification in a variety of organisms. See, for example, United States Patent Publications 20030232410; 20050208489; 20050026157; 20050064474; 20060188987; 20060063231; and International Publication WO 07/014275.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474 and 20060188987 and in U.S. application Ser. No. 11/805,850 (filed May 23, 2007), the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication No. 2008/0131962, the disclosure of which is incorporated by reference in its entirety for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KM” domains, respectively). (See US Publication No. 2011/0201055).

Engineered cleavage half-domains described herein can be prepared using any suitable method, for example, by site-directed mutagenesis of wild-type cleavage half-domains (Fok I) as described in U.S. Patent Publication Nos. 20050064474 and 20080131962.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014275. Expression of the nuclease may be under the control of a constitutive promoter or an inducible promoter, for example the galactokinase promoter which is activated (de-repressed) in the presence of raffinose and/or galactose and repressed in presence of glucose.

Cold Shock Conditions

The methods described herein involve subjecting the differentiated cells to a period of cold shock before reprogramming into iPSCs or prior to transdifferentiation. In certain embodiments, the differentiated cells are also genetically modified (e.g., using one or more nucleases) before, during or after introduction of the cold-shock treatment. Typically, the cells are cold-shocked following introduction of (e.g., transfection with) genomic modification (e.g., with a nuclease and/or donor nucleotide). The cells may be cold-shocked within minutes after transfection or may be maintained at 37° C. for a short period of time (1 day for example) prior to shifting to the cooler temperature. Cold shock can also be performed immediately after the treatment for the production of iPSC (i.e. lentiviral delivery of the Yamanaka factors, (see Takahashi and Yamanaka, ibid) or other methods for producing iPS cells) without any nuclease delivery, to increase the efficiency of iPS production. Cold shock can also be performed immediately before, during or after treatment with lineage specific factors to induce transdifferentiation.

The period of time for which the cells are cold shocked can vary from hours to days. In certain embodiments, the cells are cold-shocked for between 1 and 4 days (including 1, 2, 3, 4 day periods of any hour increment therebetween). It will be apparent that the period of cold shock will also vary depending on the cell type into which the nuclease is introduced.

Likewise, the temperature at which the cells are cold-shocked is any temperature that reduces cell division, but at which, when present, the nuclease(s) is (are) expressed and/or active. Suitable temperatures will vary depending on the host cell type. For mammalian cells, cold shock temperatures include, but are not limited to, 33° C., 32° C., 31° C., 30° C., 29° C., 28° C., 27° C., and even lower. For zebrafish and plant cells, the cold shock temperatures will typically be lower, for example 26° C., 25° C., 24° C., 23° C., 22° C., 21° C., 20° C., 19° C., 18° C. or even lower. Furthermore, the temperature can vary during the period of cold-shocking, so long as it remains low enough so that the cells are not dividing or are dividing at a reduced rate. See, also, U.S. Publication No. 20110129898 incorporated by reference in its entirety herein.

Reprogramming Differentiated Cells into iPSCs

In certain embodiments, the cells are transfected (e.g., plasmids, viral vectors, etc.) with expression vector encoding various stem cell-associated factors. See, e.g., Takahashi & Yamanaka (2006) Cell 126:663-676 or Yu et al. (2007) Science 318(5858):1917-1920. Transfected genes thought to be iPSC factors can include, for example, the master transcriptional regulators Oct-3/4 (PouSf1) and Sox2 and, optionally, c-Myc, and Klf4. The cells can be cold shocked at the time of the delivery of the stem cell associated factors, or they can be subjected to the cold shock immediately after delivery. If the cells are to be modified with ZFNs or TALENs, the cold shock may be performed after nuclease treatment, prior to the treatment to produce the iPSC. It also may be performed during the period for nuclease cleavage, or after delivery of the iPSC factors. After approximately 3-4 weeks, small numbers of transfected cells begin to become morphologically and biochemically similar to pluripotent stem cells, and are typically isolated through morphological selection, doubling time, reporter gene and/or antibiotic selection (e.g., antibiotic selection of Fbx15+ cells).

Alternatively, iPSCs (also referred to as protein-induced pluripotent stem cells or piPSCs) can be generated without introducing of genetic material, for example by repeated treatment of the cells with reprogramming proteins. Zhou et al. (2009) Cell Stem Cell 4:381-384.

During transdifferentiation, cold shock can be administered prior, during or immediately after the delivery of the lineage specific factors. If the cells are to be modified by nuclease treatment, the cold shock may be administered during the period for nuclease cleavage, or immediately prior to, during, or immediately after delivery of the lineage specific factors. The factors can be cloned into an inducible expression system (e.g., using lentiviral delivery of lineage specific factors under the control of the Tet operator such that expression is induced upon exposure of the cells to doxycycline). Cells are cultured continuously in the presence of the doxycycline, and are monitored for induction of lineage specific markers (for example, in the case of neuronal cells, they can be monitored for the production of Tuj1).

Kits

Also provided are kits for performing any of the above methods. The kits typically contain polynucleotides encoding one or more nucleases and/or donor polynucleotides as described herein as well as instructions for cold-shocking the cells into which the nucleases and/or donor polynucleotide are introduced. The kits can also contain cells, buffers for transformation of cells, culture media for cells, and/or buffers for performing assays. Typically, the kits also contain a label which includes any material such as instructions, packaging or advertising leaflet that is attached to or otherwise accompanies the other components of the kit.

Applications

Increasing the yield of iPSCs and transdifferentiated cells, including iPSCs or transdifferentiated cells with genomic modifications, has both basic research and clinical applications. For example, iPSCs derived from subjects with particular disease states can be used to create cells for disease modeling, drug toxicity screening, discovery, gene therapy and cell replacement (transplantation) therapies. Non-limiting examples of disease states include neurological disorders (e.g., Alzheimers, ALS, Parkinson's disease, etc.), diabetes, sickle cell anemia, hemophilia A, liver disease, pancreatic disease, heart disease (e.g., ischemic heart disease), cancers (solid tumors, blood cancers, etc.) and the like. See, e.g., Chun et al. (2010) Int J Biol Sci. 6(7): 796-805; Nelson et al. (2010) Stem Cells Cloning January 1; 3:29-37.

Furthermore, modified iPSCs or transdifferentiated cells into which a sequence has been integrated as described herein can be used as a cellular vehicle for protein-supplement gene therapy and/or to direct the stem cells into particular lineages. See, e.g., International Patent Publication WO 2010/117464, the disclosure of which is incorporated by reference in its entirety herein. Similarly, iPSCs or transdifferentiated cells containing nuclease-modified genomes may be used in therapeutic applications, for example by deleting a receptor for a virus (see United States Publication 20080159996) or for a growth factor (see United States Publication 20080188000). Such cells may be then re-introduced into a mammal to carry out a therapeutic effect.

Additionally, methods and compositions described herein may be used in the construction of transgenic animals. Genomic modifications (via NHEJ or additions and/or deletions) may be introduced in embryos, and then these genomically modified embryos may be used to create transgenic animals using any known suitable method.

Methods and compositions described herein are also used in kits suitable as described above.

The following Examples relate to exemplary embodiments of the present disclosure in which the nuclease comprises a zinc finger nuclease (ZFN). It will be appreciated that this is for purposes of exemplification only and that other nucleases can be used, for instance homing endonucleases (meganucleases) with engineered DNA-binding domains and/or fusions of naturally occurring of engineered homing endonucleases (meganucleases) DNA-binding domains and heterologous cleavage domains or TAL-effector domain nuclease fusion proteins.

### EXAMPLES

### Example 1

### Increased Production of iPSCs from Nuclease-Treated Fibroblasts

Fibroblasts are isolated from a human or mouse subject using standard techniques. Similarly, ZFNs targeted to a gene of choice in the fibroblasts are designed and incorporated into plasmids or adenoviral vectors as described in Urnov et al. (2005) Nature 435(7042):646-651, Perez et al (2008) Nature Biotechnology 26(7): 808-816, and U.S. Patent Publication 20080299580.

Expression vectors encoding the ZFNs are introduced into the fibroblasts by any suitable method. Immediately following transfection, the cells are divided into two different flasks and grown in appropriate medium at either 30° C. or 37° C. for 1, 2, 3 or 4 days.

Subsequently, pools of ZFN-treated cells or cells exhibiting the desired genomic modifications are reprogrammed into iPSCs using standard techniques, for example as described in Takahashi & Yamanaka (2006) Cell 126:663-676 or Yu et al. (2007) Science 318(5858):1917-1920. Fibroblasts subjected to nuclease-mediated modification cold shock generate more iPSCs than fibroblasts cultured and modified at 37° C.

### Example 2

### Increased Production of iPSCs from Fibroblasts

Fibroblasts are isolated from a human or mouse subject using standard techniques. Cultured fibroblasts are then reprogrammed into iPSCs using standard techniques, for example as described in Takahashi & Yamanaka (2006) Cell 126:663-676 or Yu et al. (2007) Science 318(5858):1917-1920. Cells are submitted to a cold shock prior, during or immediately following transduction of iPS factors. Fibroblasts subjected to the cold shock generate more iPSCs than fibroblasts cultured and modified at 37° C. Mouse embryonic fibroblasts (MEF, feeder layer) are grown according to distributor's instructions (e.g. PMEF-CFL from Millipore) and used with the STEMCCA Cre-excisable constitutive polycistronic (OSKM) lentivirus reprogramming kit followed according to manufacturer's directions (Millipore SCR531). Human fibroblasts, are split into four groups of cells, in triplicate and then the colonies formed in each well are counted. The conditions for the four groups are as follows:
4 conditions in triplicate:

i) Infect cells with reprogramming lentivirus @ 37°, leave at 37°

ii) Infect at 30°, leave 3 days, transfer to 37°

iii) Infect at 37°, leave 2 days at 30°, transfer onto feeder cells, and leave at 37°

iv) Infect at 37°, transfer on feeder cells, then transfer to 30°.
Prepare MEFs on Day 6.
Day 7: replate on MEFs (2e4 cells)
Day 13: Feeders added.
Colonies are then counted in each well and demonstrate that the transient hypothermic shock leads to an increased number of iPSC.

### Example 3

### Increased Efficiency of Transdifferentiation from Fibroblasts to Neurons

Using the protocol described in Vierbuchen et al. (2010) Nature 463(7284):1035-41, fibroblasts are submitted to the cold shock prior to, during or immediately after transduction with neural-specific factors. Fibroblasts subjected to the cold shock generate more neurons than fibroblasts cultured at 37° C.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of producing induced pluripotent stem cells (iPSCs) from differentiated somatic cells, the method comprising
(a) subjecting the differentiated somatic cells to cold shock conditions; and
(b) culturing the differentiated somatic cold-shocked cells under conditions such that iPSCs are produced.

2. The method of claim 1, wherein the differentiated somatic cells are selected from the group consisting of fibroblasts, keratinocytes, peripheral blood cells and astrocytes.

3. The method of claim 1, where the differentiated somatic cells are genetically modified.

4. The method of claim 3, wherein the genetic modification is mediated by a nuclease and further wherein the nuclease is introduced into the differentiated somatic cells prior to the cold shock conditions.

5. The method of claim 3, wherein the genetic modification is mediated by a nuclease and further wherein the nuclease is introduced into the differentiated somatic cells during cold shock conditions.

6. The method of claim 5, further comprising providing an exogenous sequence to the cells under conditions such that the nuclease mediates targeted integration of the exogenous sequence into the genome of the cells, thereby genetically modifying the differentiated somatic cells.

7. The method of claim 6, wherein the exogenous sequence comprises a reporter gene.

8. The method of claim 7, further comprising the step of isolating the cells expressing the reporter gene.

9. The method of claim 4, wherein the modification comprises a deletion.

10. A kit for increasing the yield of iPSCs, the kit comprising,
one or more nucleases and
instructions for introducing the nucleases into differentiated somatic cells and cold shocking the cells to increase yield of iPSCs.
